(19) United States
(12) Patent Application Publication
(10) Pub. No.: US 2003/0143176 A1
(43) Pub. Date: Jul. 31, 2003

(54) Compositions containing silicone oil-in-water emulsions, salts, alcohols and solvents

(76) Inventors: Yihan Liu, Midland, MI (US); Judith Mervane Vincent, Midland, MI (US)

Correspondence Address: Dow Corning Corporation, Intellectual Property Department, P.O. Box 994, Midland, MI, 48686-0994, US

(21) Appl. No.: 10/055,151

(22) Filed: Jan. 25, 2002

(51) Int. Cl. A61K 7/06; A61K 7/11

(52) U.S. Cl. 424/70.12

(57) ABSTRACT

Silicone oil-in-water emulsions are prepared using a silicone polyether as the surfactant, and the silicone oil-in-water emulsions are stable in an aqueous media in the presence of salts such as electrolytes, alcohols, solvents, and combinations thereof. The silicone oil-in-water emulsions are prepared by (i) polymerizing silicon atom containing monomers in water containing the monomer, a silicone polyether, a catalyst, and optionally an organic surfactant(s), until a silicone oil of a desired molecular weight is obtained. The silicone oil-in-water emulsions possess utility as additives in various coating products, personal care products, household care products, automotive care products, and petroleum products.

### FIELD OF THE INVENTION

This invention is directed to silicone oil-in-water (O/W) emulsions, and to certain compositions containing such O/W emulsions in combination with a salt, an alcohol, a solvent, or a combination of the salt, the alcohol, and the solvent.

### BACKGROUND OF THE INVENTION

Emulsions prepared with conventional organic surfactants are generally not stable in the presence of an alcohol or a solvent. When an ionic surfactant is used, the emulsions are not stable in the presence of salts. In fact, salts, lower alkyl alcohols, and certain organic solvents, are routinely used to break emulsions into separate phases to analyze content.

However, it has been found that when a silicone polyether is used to make a silicone oil-in-water emulsion, that the oil-in-water emulsion is stable in the presence of a salt, an alcohol, an organic solvent, or a combination thereof. Such stability is an advantage and benefit in personal care, household care, automotive care, and coating industry applications.

U.S. Pat. No. 5,443,760 (Aug. 22, 1995) is directed to oil-in-water emulsions containing silicone polyethers, but the emulsions are not prepared by emulsion polymerization.

U.S. Pat. No. 5,891,954 (Apr. 6, 1999) is directed to silicone oil-in-water emulsions prepared with silicone polyethers which are stable in the presence of an alcohol, however, the silicone polyethers are post added to silicone oil-in-water emulsions prepared by emulsion polymerization. It also fails to teach the stability of such emulsions in the presence of salts and solvents.

Copending U.S. patent application Ser. No. 09/668,959, filed Sep. 25, 2000, entitled “Compositions Containing Organic Oil-in-Water Emulsions, Salts, Alcohols, and Solvents”, assigned to the same assignee as this invention, contains subject matter similar to subject matter disclosed herein, except that in the copending application, the emulsions are limited to organic oils, i.e., oils containing no silicon atoms.

### BRIEF SUMMARY OF THE INVENTION

The invention relates to a method of making silicone oil-in-water emulsions by (i) preparing an aqueous phase containing water, a silicone polyether surfactant, and optionally one or more organic surfactants; (ii) preparing an oil phase which includes a silicon atom containing monomer; (iii) combining the aqueous phase and the oil phase; (iv) adding a polymerization catalyst to the combined phases; (v) agitating and heating the combined phases for a time sufficient to allow the silicon atom containing monomer to polymerize to form a silicone oil of a desired molecular weight; (vi) recovering a silicone oil-in-water emulsion containing the silicone oil in the oil phase of the silicone oil-in-water emulsion; and (vii) combining the silicone oil-in-water emulsion with a salt component, an alcohol component, a solvent component, or combinations thereof.

These and other features of the invention will become apparent from a consideration of the detailed description.

### DETAILED DESCRIPTION OF THE INVENTION

This invention is based on the unexpected discovery that when silicone polyethers are used to prepare silicone oil-in-water emulsions, the resulting formulations are stable in the presence of salts such as calcium chloride and aluminum sulfate; alcohols such as methanol, ethanol, propanol and isopropanol; and organic solvents such as pentane.

The silicone polyether can be the only emulsifier used in making these emulsion, or it can be used in combination with other organic type surfactants. The silicone polyether can be used to make silicone oil-in-water microemulsions which are also stable in the presence of such salts, alcohols, and solvents.

Silicone Polyether (SPE) Surfactant

The silicone polyether is generally water soluble or water dispersible. It can have a rake type structure wherein the polyoxyethylene or polyoxyethylene-polyoxypropylene copolymeric units are grafted onto the siloxane backbone, or the SPE can have an ABA block copolymeric structure wherein A represents the polyether portion and B the siloxane portion of an ABA structure.

Silicone polyethers suitable for use herein have the formula MD_{0-1,000}D′₁₋₁₀₀ M, most preferably the formula MD₀₋₅₀₀D′₁₋₅₀M, where M represents the monofunctional unit R₃SiO_{1/2}, D represents the difunctional unit R₂SiO_{2/2}, and D′ represents the difunctional unit RR′SiO_{2/2}. In these formulas, R is an alkyl group containing 1-6 carbon atoms or an aryl group, and R′ is an oxyalkylene containing moiety. The R′ groups may contain only oxyethylene (EO) units; a combination of oxyethylene (EO) and oxypropylene (PO) units; or a combination of oxyethylene (EO) units, oxypropylene (PO) units, and oxybutylene (BO) units. Preferred R′ groups include oxyalkylene units in the approximate ratio of EO₃₋₁₀₀PO₀₋₁₀₀, most preferably in the ratio EO₃₋₃₀PO₁₋₃₀.

R′ moieties typically includes a divalent radical such as —CₘH₂ₘ— where m is 2-8 for connecting the oxyalkylene portion of R′ to the siloxane backbone. Such moieties also contain a terminating radical for the oxyalkylene portion of R′ such as hydrogen, hydroxyl, or an alkyl, aryl, alkoxy, or acetoxy group.

Silicone polyethers useful herein can also be of a type having the formula M′D_{10-1,000}D′₀₋₁₀₀M′, most preferably the formula M′D10-500D′₀₋₅₀M′, wherein M′ represents the monofunctional unit R₂R′SiO_{1/2}, D represents the difunctional unit R₂SiO_{2/2}, and D′ represents the difunctional unit RR′SiO_{2/2}. In these formulas, R is an alkyl group containing 1-6 carbon atoms or an aryl group, and again R′ represents an oxyalkylene containing moiety. As noted previously, R′ groups typically contain only oxyethylene (EO) units or combinations of oxyethylene (EO) and oxypropylene (PO) units. Such R′ groups include these oxyalkylene units in the ratio EO₃₋₁₀₀PO₀₋₁₀₀, most preferably EO₃₋₃₀PO₁₋₃₀.

As also noted previously, R′ moieties typically include a divalent radical —CₘH₂ₘ— where m is 2-8 for connecting the oxyalkylene portions of R′ to the siloxane backbone. In addition, the moiety R′ contains a terminating radical for oxyalkylene portions of R′ such as hydrogen, hydroxyl, an alkyl, aryl, alkoxy, or acetoxy group.

In addition, silicone polyethers useful herein can be of a type having the formula MD_{0-1,000}D′₀₋₁₀₀D″_{1-1,00}M wherein D″ represents the difunctional unit RR″SiO_{2/2}, and R″ is an alkyl group containing 1-40 carbon atoms. If desired, R″ can also be an aryl group such as phenyl; an arylalkyl group such as benzyl; an alkaryl group such as tolyl; or R″ can represent a substituted alkyl group such as aminoalkyl, epoxyalkyl, or carboxyalkyl. M, D, D′, and R, are the same as defined above.

Table I shows some representative silicone polyethers according to such formulas, and these compositions are referred to in the accompanying Examples. The HLB (hydrophile-lipophile balance) of each silicone polyether is a value obtained by dividing the molecular weight percent of the ethylene oxide portion of each molecule by five.

TABLE I

| Silicone | | |
|---|---|---|
| Poly- | | |
| ether | Nominal Structure of the Silicone Polyether | HLB |
| A | M′D₁₃M′ where R is —CH₃ and R′ is —(CH₂)₃(EO)₁₂OH | 9.2 |
| B | MD₁₀₈D′ ₁₀M where R is —CH₃ and | 6.6 |
| | R′ is —(CH₂)₃(EO)₁₈(PO)₁₈OAc | |
| C | MD_{8.6}D′_{3.6}M where R is —CH_{3 and R′ is} | 12.3 |
| | —(CH₂)₃(EO)₁₂OH | |

Silicone Oil Component

Silicone oils are often provided as aqueous emulsions or microemulsions of a polydimethylsiloxane stabilized in the emulsion or microemulsion by one or more surfactants. The silicone oil in the aqueous emulsion or microemulsion can be a linear or branched chain siloxane fluid having a viscosity of about 100-300,000 mm²/s (cS) at 25° C. Most useful are polymers and copolymers having a viscosity in the range of about 300-60,000 mm²/s, most preferably about 350-15,000 mm²/s. A mixture of silicone oils having relatively higher and relatively lower viscosity can also be employed.

Such silicone oils, i.e., polysiloxanes, contain the characteristic difunctional repeating “D” unit:

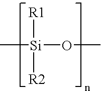

in which n is greater than 1; and R1 and R2 are each independently alkyl radicals containing 1-7 carbon atoms or a phenyl group.

Illustrative silicone oils are polydimethylsiloxane, polydiethylsiloxane, polymethylethylsiloxane, polymethylphenylsiloxane, and polydiphenylsiloxane. Preferably, the silicone oil is one that is trimethylsiloxy terminated, but it can also include those silicone oils having hydroxy endblocking units as well.

While the silicone oil can contain “D” units other than dimethylsiloxane, such as diphenyl siloxane or methylphenyl siloxane, from the standpoint of economics, polymers with dimethylsiloxane “D” units —[(CH₃)₂SiO]— are most preferred. Yet, in some instances, it might be appropriate for R1 or R2 to represent another functional group, such as an aminoalkyl, carboxyalkyl, haloalkyl, acrylate, acryloxy, acrylamide, or vinyl group, for example.

Additional and/or Optional Organic Surfactant

While the silicone polyether is capable of functioning as the sole emulsifying agent, other optional and additional organic surfactants can be included in combination with the silicone polyether surfactant, if desired.

Such other surfactant can be a nonionic, cationic, anionic, amphoteric (zwitterionic), or a mixture of such surfactants. The nonionic surfactant should be a non-silicon atom containing nonionic emulsifier. Most preferred are alcohol ethoxylates R3-(OCH2CH2)cOH, most particularly fatty alcohol ethoxylates. Fatty alcohol ethoxylates typically contain the characteristic group —(OCH₂CH₂)_{c}OH which is attached to fatty hydrocarbon residue R3 which contains about eight to about twenty carbon atoms, such as lauryl (C₁₂), cetyl (C₁₆) and stearyl (C₁₈). While the value of “c” may range from 1 to about 100, its value is typically in the range of 2 to 40.

Some examples of suitable nonionic surfactants are polyoxyethylene (4) lauryl ether, polyoxyethylene (5) lauryl ether, polyoxyethylene (23) lauryl ether, polyoxyethylene (2) cetyl ether, polyoxyethylene (10) cetyl ether, polyoxyethylene (20) cetyl ether, polyoxyethylene (2) stearyl ether, polyoxyethylene (10) stearyl ether, polyoxyethylene (20) stearyl ether, polyoxyethylene (21) stearyl ether, polyoxyethylene (100) stearyl ether, polyoxyethylene (2) oleyl ether, and polyoxyethylene (10) oleyl ether. These and other fatty alcohol ethoxylates are commercially available under names such as ALFONIC®, ARLACEL, BRIJ, GENAPOL®, LUTENSOL, NEODOL®, RENEX, SOFTANOL, SURFONIC®, TERGITOL®, TRYCOL, and VOLPO.

Cationic surfactants useful in the invention include non-silicon atom containing compounds having quaternary ammonium hydrophilic moieties in the molecule which are positively charged, such as quaternary ammonium salts represented by R4R5R6R7N⁺X⁻ where R4 to R7 are alkyl groups containing 1-30 carbon atoms, or alkyl groups derived from tallow, coconut oil, or soy; and X is halogen such as chlorine or bromine, or X can be a methosulfate group. Most preferred are (i) dialkyldimethyl ammonium salts represented by R8R9N⁺(CH₃)₂X⁻, where R8 and R9 are alkyl groups containing 12-30 carbon atoms, or alkyl groups derived from tallow, coconut oil, or soy; and X is halogen or a methosulfate group; or (ii) monoalkyltrimethyl ammonium salts represented by R10N⁺(CH₃)₃X⁻ where R10 is an alkyl group containing 12-30 carbon atoms, or an alkyl group derived from tallow, coconut oil, or soy; and X is halogen or a methosulfate group.

Representative quaternary ammonium salts are dodecyltrimethyl ammonium bromide (DTAB), dodecyltrimethyl ammonium chloride, tetradecyltrimethyl ammonium bromide, tetradecyltrimethyl ammonium chloride, hexadecyltrimethyl ammonium bromide, hexadecyltrimethyl ammonium chloride, didodecyldimethyl ammonium bromide, dihexadecyldimethyl ammonium chloride, dihexadecyldimethyl ammonium bromide, dioctadecyldimethyl ammonium chloride, dieicosyldimethyl ammonium chloride, didocosyldimethyl ammonium chloride, dicoconutdimethyl ammonium chloride, ditallowdimethyl ammonium chloride, and ditallowdimethyl ammonium bromide. These and other quaternary ammonium salts are commercially available under names such as ADOGEN, ARQUAD, SERVAMINE, TOMAH, and VARIQUAT.

Examples of non-silicon atom containing anionic surfactants include sulfonic acids and their salt derivatives such as dodecylbenzene sulfonic acid (DBSA); alkali metal sulfosuccinates; sulfonated glyceryl esters of fatty acids such as sulfonated monoglycerides of coconut oil acids; salts of sulfonated monovalent alcohol esters such as sodium oleyl isothionate; amides of amino sulfonic acids such as the sodium salt of oleyl methyl tauride; sulfonated products of fatty acid nitriles such as palmitonitrile sulfonate; sulfonated aromatic hydrocarbons such as sodium alpha-naphthalene monosulfonate; condensation products of naphthalene sulfonic acids with formaldehyde; sodium octahydro anthracene sulfonate; alkali metal alkyl sulfates such as sodium lauryl (dodecyl) sulfate (SDS); ether sulfates having alkyl groups of eight or more carbon atoms; and alkylaryl sulfonates having one or more alkyl groups of eight or more carbon atoms.

Commercial anionic surfactants useful in this invention include triethanolamine linear alkyl sulfonate sold under the name BIO-SOFT N-300 by the Stepan Company, Northfield, Ill.; sulfates sold under the name POLYSTEP by the Stepan Company; and sodium n-hexadecyl diphenyloxide disulfonate sold under the name DOWFAX 8390 by The Dow Chemical Company, Midland, Mich.

Surfactants classified as amphoteric or zwitterionic include cocoamphocarboxy glycinate, cocoamphocarboxy propionate, cocobetaine, N-cocamidopropyldimethyl glycine, and N-lauryl-N-carboxymethyl-N-(2-hydroxyethyl)ethylene diamine. Other suitable amphoteric surfactants include the quaternary cycloimidates, betaines, and sultaines.

The betaines have the structure R11R12R13N⁺(CH₂)pCOO⁻ wherein R11 is an alkyl group having about twelve to eighteen carbon atoms or a mixture thereof, R12 and R13 are independently lower alkyl groups having one to three carbon atoms, and p is an integer from one to four. Specific betaines are α-(tetradecyldimethylammonio)acetate, β-(hexadecyldiethylammonio)propionate, and γ-(dodecyldimethylammonio)butyrate.

The sultaines have the structure R11R12R13N⁺(CH₂)pSO₃⁻ wherein R11, R12, R13, and p are as defined above. Specific useful sultaines are 3-(dodecyldimethylammonio)-propane-1-sulfonate, and 3-(tetradecyldimethylammonio)ethane-1-sulfonate.

Representative amphoteric surfactants are products sold under the names MIRATAINE® by Rhone-Poulenc Incorporated, Cranberry, N.J.; and TEGO BETAINE by Goldschmidt Chemical Corporation, Hopewell, Va. Imidazoline and imidazoline derivatives sold under the name MIRANOL® by Rhone-Poulenc Incorporated, Cranberry, N.J. may also be employed.

Salt Component

As used herein, the term “salt” is intended to mean an inorganic salt or an organic salt, including compounds commonly referred to as electrolytes. Some examples of suitable inorganic salts include calcium chloride, magnesium sulfate, magnesium chloride, sodium sulfate, sodium thiosulfate, sodium chloride, sodium phosphate, ammonium chloride, ammonium carbonate, iron sulfate, aluminum sulfate, aluminum chloride, aluminum chlorohydrate, aluminum sesquichlorohydrate, aluminum dichlorohydrate, aluminum zirconium tetrachorohydrex glycine, aluminum zirconium trichlorohydrate, aluminum zirconium tetrachlorohydrate, aluminum zirconium pentachlorohydrate, and aluminum zirconium octachlorohydrate.

Some examples of suitable organic salts include sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, sodium oxalate, sodium phenate, sodium propionate, sodium saccharin, sodium salicylate, sodium sarcosinate, sodium toluene sulfonate, magnesium aspartate, calcium propionate, calcium saccharin, calcium d-saccharate, calcium thioglycolate, aluminum caprylate, aluminum citrate, aluminum diacetate, aluminum glycinate, aluminum lactate, aluminum methionate, aluminum phenosulfonate, potassium aspartate, potassium biphthalate, potassium bitartrate, potassium glycosulfate, potassium sorbate, potassium thioglycolate, potassium toluene sulfonate, and magnesium lactate.

Alcohol Component

The term “alcohol” as used herein is intended to mean a lower alkyl alcohol such as ethanol. Examples of some other appropriate lower alkyl alcohols which can be used are methyl alcohol, propyl alcohol, isopropyl alcohol, butyl alcohol, and isobutyl alcohol. Generally, these lower alkyl alcohols will contain one to about four carbon atoms.

Solvent Component

Solvents which can be used herein include alkanes with generally less than about 16 carbon atoms such as pentane and hexane; ketones such as acetone, methyl ethyl ketone, methyl n-butyl ketone, and methyl amyl ketone; aromatic compounds such as benzene, toluene, and ethylbenzene; esters such as ethyl acetate, isopropyl acetate, methyl acetoacetate, and isobutyl isobutyrate; ethers such as ethyl ether, butyl ethyl ether, isopentyl ether, propylene oxide, and tetrahydrofuran; glycols such as ethylene glycol, propylene glycol, and diethylene glycol; and chlorinated hydrocarbons such as methylene chloride, chloroform, carbon tetrachloride, ethyl chloride, and chlorobenzene.

Optional Components

Since emulsions are susceptible to microbiological contamination, a preservative may be required as an optional component of the emulsion, and some representative compounds which can be used include formaldehyde, salicylic acid, phenoxyethanol, DMDM hydantoin (1,3-dimethylol-5,5-dimethyl hydantoin), 5-bromo-5-nitro-1,3-dioxane, methyl paraben, propyl paraben, sorbic acid, imidazolidinyl urea sold under the name GERMALL® II by Sutton Laboratories, Chatham, N.J., sodium benzoate, 5-chloro-2-methyl-4-isothiazolin-3-one sold under the name KATHON CG by Rohm & Haas Company, Philadelphia, Pa., and iodopropynl butyl carbamate sold under the name GLYCACIL® L by Lonza Incorporated, Fair Lawn, N.J.

A freeze/thaw stabilizer can be included as an optional component of the emulsion including compounds such as ethylene glycol, propylene glycol, glycerol, trimethylene glycol.

Another optional component is a corrosion inhibitor such as an alkanolamine, an inorganic phosphate such as zinc dithiophosphate, an inorganic phosphonate, an inorganic nitrite such as sodium nitrite, a silicate, a siliconate, an alkyl phosphate amine, a succinic anhydride such as dodecenyl succinic anhydride, an amine succinate, or an alkaline earth sulfonate such as sodium sulfonate or calcium sulfonate.

Compositions

Compositions capable of being prepared according to the concept of the present invention will generally contain one or more of the salt component, the alcohol component, or the solvent component, in amounts as follows:

(i) 1-30 percent by weight of the salt component,

(ii) 1-80 percent by weight of the alcohol component,

(iii) 1-80 percent by weight of the solvent component,

and (iv) 10-90 percent by weight of the silicone oil-in-water emulsion. The silicone oil-in-water emulsion in turn will generally contain 5-80 percent by weight of the silicone oil, 0.1-20 percent by weight of the surfactant(s), and the balance to 100 percent by weight being water.

When it is desired to include an optional component in the composition, 0.01-0.1 percent by weight of each optional component, i.e., preservative, freeze/thaw stabilizer, or corrosion inhibitor, can be added to the composition. Such compositions can generally be prepared at room temperature using simple propeller mixers, turbine-type mixers, Brookfield counter-rotating mixers, or homogenizing mixers. No special equipment or processing conditions are generally required.

Emulsion Preparation

Mechanical preparation of an emulsion involves mixing water, one or more surfactants, and an oil, and homogenizing the mixture using a laboratory homogenizer or other device for applying vigorous agitation. Silicone polyethers can be incorporated in mechanical processes as the sole emulsifier, or they can be used with co-surfactants such as other organic surfactants. Silicone polyethers can also be post-added to previously prepared emulsions. Mechanical processes are described in U.S. Pat. No. 5,017,221 (May 21, 1991) and in EP 463431 (Jan. 2, 1992), for example.

Emulsions prepared by emulsion polymerization involve mixing water, surfactant(s), and silicon atom containing monomers, with a polymerization catalyst. The mixture is agitated until essentially all of the silicon atom containing monomer is reacted and consumed, and a stable emulsion is formed. The silicone polyether is generally incorporated before polymerization occurs, i.e., before the catalyst is added. Processes of emulsion polymerization are described in U.S. Pat. Nos. 5,891,954 (Apr. 6, 1999) and 6,316,541 (Nov. 13, 2001), which are considered incorporated herein by reference.

Silicon Atom Containing Monomer

The polymerization reaction occurring during emulsion polymerization involves the opening of the cyclic siloxane ring of the monomer, using an acid or base catalyst, and in the presence of water. Upon opening of the ring, polysiloxanes oligomers with terminal hydroxy groups are formed. These polysiloxane oligomers then react with one another or with other siloxane reactants that may be present in the reaction medium, through a condensation reaction, to form polysiloxane polymers or polysiloxane copolymers, i.e., silicone oils. Monomers useful in the method of this invention are those generally insoluble in water which can be readily polymerized using emulsion polymerization. Preferred monomers can be represented by the formula

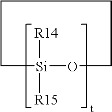

wherein R14 and R15 are each independently selected from saturated or unsaturated alkyl groups containing 1-6 carbon atoms; aryl groups containing 6-10 carbon atoms; and wherein R14 and R15 optionally can contain functional groups which are unreactive in the ring opening and polymerization reaction. Generally, t has a value of 3-7.

In particular, R14 and R15 can be represented by groups such as methyl, ethyl, propyl, phenyl, allyl, or vinyl groups; or R14 and R15 can represent groups such as —R16-F, wherein R16 is an alkylene group with 1-6 carbon atom or an arylene group with 6-10 carbon atoms, and F is a functional group such as an amine, diamine, halogen, carboxy, or mercapto group. If desired, R14 and R15 can also represent groups such as —R16-F2-R17 wherein R17 is the same as defined above for R14 and R15, and F2 is a non-carbon atom such as oxygen, nitrogen, or sulfur. Some monomers particularly preferred for this invention can be exemplified by hexamethylcyclotrisiloxane, octamethylcyclotetrasiloxane, decamethylcyclopentasiloxane, tetramethyltetravinylcyclotetrasiloxane, and tetramethyltetraphenylcyclotetrasiloxane.

It's possible to produce copolymers via the emulsion polymerization reaction by having present in the reaction medium, a small portion of other types of silicon atom containing monomers. Such other monomers can be any silicon atom containing composition having hydrolyzable or silanol groups, capable of being polymerized using emulsion polymerization. Some examples of these other monomers include amine functional silanes, vinyl functional silanes, halogen alkyl functional silanes, and hydroxy endblocked polysiloxanes. In particular, they include silanol terminated polydimethysiloxanes with a degree of polymerization (DP) of 1-7; methyltrimethoxysilane; ethyltrimethoxysilane; propyltrimethoxysilane; phenyltrimethoxysilane; methylphenyldimethoxysilane; N-(2-aminoethyl)-3-aminopropyltrimethoxysilane; tetraethoxysilane; trimethoxyvinylsilane; tris-(2-methoxyethoxy)vinylsilane; and 3-chloropropryltrimethoxysilane.

In particular, the preferred method of making silicone oil-in-water emulsions according to the invention involves (i) preparing an aqueous phase containing water, a silicone polyether surfactant, and optionally one or more organic surfactants; (ii) preparing an oil phase comprising a silicon atom containing monomer polymerizble to form a silicone oil of a desired molecular weight; (iii) combining the aqueous phase and the oil phase, and applying shear; (iv) adding a polymerization catalyst to the combined phases; (v) heating and agitating the combined phases for a time sufficient to allow the silicon atom containing monomer to polymerize and to form an oil-in-water emulsion containing the silicone oil of desired molecular weight; and (vi) recovering the oil-in-water emulsion containing the silicone oil.

Such emulsions typically have their pH adjusted to 6-7.5, and in general contain 5-80 percent by weight of the silicone oil, preferably 20-60 percent; 0.1-20 percent by weight of the surfactant(s), preferably 0.1-10 percent; and the balance to 100 percent by weight of water, based on the weight of the emulsion.

A variety of types of emulsions can be prepared according to this process. For example, microemulsions can be prepared in which the silicone oils are present as particles having a diameter of less than 140 nanometer (0.14 micrometer), preferably less than 50 nanometer (0.05 micrometer). In the case offine emulsions, they are present as particles with diameters of 140-300 nanometer (0.14-0.30 micrometer). In standard emulsions, on the other hand, they are present as particles with diameters greater than 300 nanometer (0.30 micrometer).

Stability Measure

Emulsion stability was evaluated by visual observation. A stable emulsion was one that did not evidence any separation or creaming effect. An unstable emulsion was indicated by the emulsion separating into an oil-rich and a water-rich layer or sedimentation. Only the initial stability was determined in the examples. However, many of the formulations showed long term stability of the order of weeks and months.

### EXAMPLES

The following examples are set forth in order to illustrate this invention in more detail.

### Example I

### Mechanical Emulsification

A first portion used as Part A was prepared in a vial by adding to the vial 1.5 gram of stearic acid, 0.5 gram of glyceryl stearate and PEG-100 stearate nonionic surfactant sold under the tradename Arlacel 165, and 5 gram of decamethylcyclopentasiloxane. The contents of the vial were mixed and heated to about 80° C. to melt the surfactant. A second portion used as Part B was prepared in a 100 ml container by adding to the container 40.07 gram of deionized water, 0.93 gram of a solution containing triethanolamine as 85 percent active in water, and 2 gram of the Silicone Polyether A shown above in Table I. The contents of the container were mixed while heating to 40° C. The contents of Part A were poured slowly into Part B while continuing heating and mixing with a laboratory mixer rotating at 350 RPM. The final composition was agitated at 350 RPM for an additional 30 minutes at 40° C. An emulsion was formed and is referred to hereafter as Emulsion I.

One gram of calcium chloride salt was added to 2 gram of Emulsion I and mixed. The composition was stable. Two gram of methanol alcohol was added to 2 grams Emulsion I and shaken. The composition was stable initially but after 3 days showed partial agglomeration. Two gram of isopropanol alcohol was added to 2 gram of Emulsion I and shaken. The composition was stable initially but after 3 days showed partial agglomeration. Two gram of pentane solvent was added to 2 grams of Emulsion I and shaken. The composition separated into a clear top phase of pentane and a bottom phase of Emulsion I. The emulsion stayed intact without being extracted by the pentane phase for more than 3 days. When Silicone Polyether A was omitted from Part B in the process of making Emulsion I, it was found that the addition to Emulsion I of the same proportions of calcium chloride salt, methanol alcohol, and isopropanol alcohol, resulted in Emulsion I breaking instantly; while addition of the pentane solvent to Emulsion I extracted the silicone oil from Emulsion I.

### Example II

### Mechanical Emulsification

In a cream jar, there were combined 30 gram of deionized water, 7.5 gram of Silicone Polyether A, and 12.5 gram of a 350 centistoke (mm²/sec) polydimethylsiloxane silicone oil. The composition was sonicated with a soniprobe in a pulsed mode for one minute. An emulsion was formed and is referred to hereafter as Emulsion II. Five gram of Emulsion II was diluted with 15 ml of isopropanol alcohol and shaken. The composition was stable.

### Example III

### Mechanical Emulsification

Using the same procedure as in Example II, and by replacing Silicone Polyether A with the Silicone Polyether B shown in Table I above, another emulsion was formed, referred to hereafter as Emulsion III. 15 ml of methanol alcohol was added to 5 gram of Emulsion III and shaken. The composition was stable. The same results were obtained when ethanol alcohol or isopropanol alcohol were used in place of methanol alcohol. 15 ml of pentane solvent was added to 5 gram of Emulsion III and shaken. The composition separated into a clear top phase of pentane solvent and a bottom phase of Emulsion III. Emulsion III stayed intact without being extracted by the pentane solvent. The addition of 0.25 gram of calcium chloride salt, 12.5 ml of methanol alcohol, and 12.5 ml of pentane solvent, to 5 gram of Emulsion III, followed by shaking, produced a homogeneous emulsion showing no evidence of phase separation.

### Example IV

### Emulsion Polymerization

An oil-in-water microemulsion containing as the silicone oil, a linear hydroxy-terminated polydimethylsiloxane, was prepared by emulsion polymerization. According to the procedure, there was added to a 500 ml round bottom flask, 123.17 gram of deionized water, 28.21 gram of dodecylbenzenesulfonic acid, and 34 gram of Silicone Polyether A. The flask contents were stirred at 300 RPM while being heated at 70° C. After the surfactant had dispersed, 75 gram of octamethylcyclotetrasiloxane monomer was fed to the mixture over a 20 minutes interval and at a constant rate. The reaction was maintained at 70° C. and agitated at 300 RPM for a period of time of 5 hours measured from initiation of the monomer feed. To the mixture was then added an additional amount of 15.03 gram of Silicone Polyether A and 39 gram of deionized water. The mixture was cooled to room temperature. The reaction mixture was neutralized using 17.5 gram of triethanolamine solution with an active content of 85 percent in water. The microemulsion was preserved by the addition of 0.3 gram of Kathon CG preservative, and is referred to hereafter as Microemulsion IV. It was transparent and had a particle size of 34 nanometer. To 2 gram of Microemulsion IV was added one gram of calcium chloride salt and mixed. The composition was stable. To 2 gram of Microemulsion IV was added 2 gram of methanol alcohol and shaken. The composition became milky but remained homogenous and showed no evidence of phase separation.

### Example V

### Emulsion Polymerization

Another oil-in-water microemulsion, containing as the silicone oil a lightly crosslinked polydimethylsiloxane, was prepared by emulsion polymerization. According to the procedure, there was added to a 500 ml round bottom flask, 150.18 gram of deionized water, 28.1 gram of dodecylbenzenesulfonic acid, and 5.6 gram of the Silicone Polyether C shown above in Table I. The contents of the flask were stirred at 300 RPM while being heated to about 85° C. After the surfactant had been dispersed, 1.06 gram of crosslinking monomer tetraethoxysilane was added to the flask. There was fed to the flask, 86.97 gram of octamethylcyclotetrasiloxane monomer over an interval of 30 minutes at a constant rate. The reaction was maintained at about 85° C. and agitated at 300 RPM for another period of about 5 hours. To the flask contents was then added another 17.58 gram portion of Silicone Polyether C and an additional portion of 43.42 gram of deionized water. The flask was cooled to room temperature. To the flask was then added 18.21 gram of triethanolamine as a solution of 85 percent of the active in water, to neutralize the reaction. 0.36 gram of Kathon CG was added for preservation of the microemulsion.

The microemulsion, hereafter referred to as Microemulsion V, was translucent and had a particle size of 57 nanometer. Microemulsion V remained stable for more than 6 months. To 5 grams of Microemulsion V was added one gram of aluminum sulfate salt and mixed. The composition was stable and clear for more than 6 months. To 5 grams of Microemulsion V, was added 15 ml of methanol alcohol and shaken. The composition became milky but remained homogeneous and showed no evidence of phase separation for more than 6 months. To 5 grams of Microemulsion V was added 15 ml of ethanol alcohol and shaken. The composition became slightly milky but remained homogeneous, and showed no evidence of phase separation for more than 6 months. To 5 grams of Microemulsion V was added 15 ml of isopropanol alcohol and shaken. The composition became slightly milky but remained homogeneous, and showed no evidence of phase separation for more than 6 months. The clarity of the isopropanol alcohol diluted composition was similar in clarity obtained when water was used to dilute the microemulsion.

Emulsions and microemulsions prepared according to this invention are useful in paper coating, textile coating, personal care, household care, automotive care, and petroleum industry, applications for delivering silicone polymers to various surfaces and substrates. For example, in personal care, they can be used in underarm products such as antiperspirants and deodorants, hair care products such as styling aids, and in products used in the care of skin.

Other variations may be made in compounds, compositions, and methods described herein without departing from the essential features of the invention. The embodiments of the invention specifically illustrated herein are exemplary only and not intended as limitations on their scope except as defined in the appended claims.

1. A method of making a silicone oil-in-water emulsion comprising (i) preparing an aqueous phase containing water, a silicone polyether surfactant, and optionally one or more organic surfactants; (ii) preparing an oil phase comprising a silicon atom containing monomer polymerizable to a silicone oil of a desired molecular weight; (iii) combining the aqueous phase and the oil phase; (iv) adding a polymerization catalyst; (v) heating and agitating the combined phases for a time sufficient to allow the silicon atom containing monomer to polymerize to a silicone oil having the desired molecular weight; and (vi) recovering a silicone oil-in-water emulsion containing the silicone oil of the desired molecular weight.

2. A silicone oil-in-water emulsion prepared by the method according to claim 1.

3. A composition comprising the silicone oil-in-water emulsion according to claim 2 and a component selected from the group consisting of salts, alcohols, solvents, and mixtures of salts, alcohols, and solvents.

4. A composition according to claim 3 in which the component is a salt, and the salt is an inorganic salt or an organic salt selected from the group consisting of calcium chloride, magnesium sulfate, magnesium chloride, sodium sulfate, sodium thiosulfate, sodium chloride, sodium phosphate, ammonium chloride, ammonium carbonate, iron sulfate, aluminum sulfate, aluminum chloride, aluminum chlorohydrate, aluminum sesquichlorohydrate, aluminum dichlorohydrate, aluminum zirconium tetrachorohydrex glycine, aluminum zirconium trichlorohydrate, aluminum zirconium tetrachlorohydrate, aluminum zirconium pentachlorohydrate, aluminum zirconium octachlorohydrate, sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, sodium oxalate, sodium phenate, sodium propionate, sodium saccharin, sodium salicylate, sodium sarcosinate, sodium toluene sulfonate, magnesium aspartate, calcium propionate, calcium saccharin, calcium d-saccharate, calcium thioglycolate, aluminum caprylate, aluminum citrate, aluminum diacetate, aluminum glycinate, aluminum lactate, aluminum methionate, aluminum phenosulfonate, potassium aspartate, potassium biphthalate, potassium bitartrate, potassium glycosulfate, potassium sorbate, potassium thioglycolate, potassium toluene sulfonate, and magnesium lactate.

5. A composition according to claim 3 in which the component is an alcohol, and the alcohol component is a lower alkyl alcohol containing one to about four carbon atoms.

6. A composition according to claim 3 in which the component is a solvent, and the solvent component is an alkane containing less than about 16 carbon atoms, a ketone, an aromatic compound, an ester, an ether, a glycol, or a chlorinated hydrocarbon.

7. A product containing the silicone oil-in-water emulsion according to claim 2, the product being selected from the group consisting of coating products, personal care products, household care products, automotive care products, and petroleum products.

8. A method of treating the underarm, hair, or skin of the human body comprising applying to the underarm, hair, or skin of the human body, a personal care product according to claim 7.
